SEQUENCE LISTING (3 sequences)

SEQ ID NO: 1 - DNA, 31 bp, Artificial Sequence
  Linker
tccatctttccgcatcaacgaatatgttagc

SEQ ID NO: 2 - DNA, 33 bp, Artificial Sequence
  Linker
tctccatctttccgcatcaacgaatatgttagc

SEQ ID NO: 3 - DNA, 30 bp, Artificial Sequence
  Linker
ccatctttccgcatcaacgaatatgttagc

(19) United States
(12) Patent Application Publication
(10) Pub. No.: US 2025/0003958 A1
(43) Pub. Date: Jan. 2, 2025

(54) REAGENT FOR PREPARING A CELL SAMPLE

(71) Applicant: SONY GROUP CORPORATION, TOKYO (JP)

(72) Inventors: KENZO MACHIDA, TOKYO (JP); AYA FUCHIGAMI, TOKYO (JP); YOSHIHITO HAYASHI, TOKYO (JP)

(21) Appl. No.: 18/578,564

(22) PCT Filed: Feb. 28, 2022
(86) PCT No.: PCT/JP2022/008360

§ 371 Date: Jan. 11, 2024

(30) Foreign Application Priority Data
Jul. 28, 2021 (JP) 2021-123441

(51) Int. Cl. G01N 33/543; G01N 33/58; G01N 33/68

(52) U.S. Cl. CPC G01N 33/54326; G01N 33/582; G01N 33/6854

(57) ABSTRACT

Disclosed is a reagent for preparing a cell sample, the reagent containing a solid support and a trapping substance that is linked to the solid support by a cleavable linker and traps a target cell. In the reagent for preparing a cell sample, a fluorescent dye is bound to a site on the linker closer to the trapping substance than the cleavage position in the linker, or a site on the trapping substance except for a binding site to the target cell in the trapping substance. The reagent for preparing a cell sample is useful for enriching a target cell using an antibody-modified solid support and subsequent staining for separating by a cell sorter, at the same time.

### TECHNICAL FIELD

The present technology relates to a reagent for preparing a cell sample

### BACKGROUND ART

As a process for collecting immune cells required for producing a cell-based medicine for use in, e.g., genetically-modified T cell therapy (CAR T therapy), a method using antibody-modified magnetic beads is frequently used. In the method using magnetic beads, it is possible to treat a large amount of sample at a time but small cell subsets cannot be separated in a single treatment. Furthermore, since the beads become no longer required in the subsequent production process, the beads must be removed from the cells.

In order to separate a desired cell subset, it is necessary to repeat a collection method using beads or use a cell sorter. Repeating the method using beads may reduce a collection rate or may decrease viability due to a longer process time. On the other hand, for separating cells by a cell sorter, it is necessary to perform a step of staining the cells with an appropriate fluorescent dye.

Patent Document 1 discloses a technique for cleaving a bond formed between a support (e.g., beads) on which denatured streptavidin is immobilized and a compound (e.g., antibody) modified with denatured biotin at an appropriate time. In this technique, the strong binding force between streptavidin not denatured and biotin is appropriately weakened by replacing them with denatured streptavidin such as nitrostreptavidin and a denatured biotin such as desthiobiotin, respectively. In this manner, at the time point when the beads become no longer required after cells are collected by antibody-modified beads, the beads can be separated from the cells.

Patent Document 2 and Patent Document 3 disclose a technique for reversibly staining or collecting with Strep-tag and Strep-Tactin. In this technique, multimerized Strep-Tactin and Strep-tag having a receptor-binding site are used in combination and cells are fluorescently stained. After the cells are separated by a cell sorter, biotin is added to dissociate the bond between Strep-Tactin and Strep-tag and remove a fluorescent label from the cells. Similarly, in the technique, if a magnetic bead is used in place of the fluorescent label to the multimerized Strep-Tactin, the beads can be separated from the target cells collected. If this method is repeated, separation of cells (multiple positive selections) can be performed with multiple markers, and small cell subsets can be taken out (Non-Patent Document 1).

### CITATION LIST

### Patent Document

Patent Document 1: EP2518193A2
Patent Document 2: U.S. Pat. No. 9,188,589B2
Patent Document 3: Japanese Unexamined Patent Application Publication No. 2014-529361

### Non-Patent Document

Non-Patent Document 1: PLOS ONE 7 (4): e35798

### SUMMARY OF THE INVENTION

### Problems to be Solved by the Invention

However, the collection of target cells using a solid support such as beads and the staining of the collected cells with a fluorescent label are generally performed in separate steps. Since the steps are performed on a one-by-one basis, it takes a long time for preparing a cell sample, with the result that viability may decrease.

Thus, the present technology provides a reagent for preparing a cell sample which enables enrichment of a target cell using an antibody-modified solid support and subsequent staining for separating by a cell sorter, at the same time.

### Solutions to Problems

The present technology provides a reagent for preparing a cell sample, the reagent containing a solid support and a trapping substance that is linked to the solid support by a cleavable linker and traps a target cell, in which a fluorescent dye is bound to a site on the linker closer to the trapping substance than a cleavage position in the linker or a site on the trapping substance except for a binding site to the target cell in the trapping substance.

Moreover, the present technology provides a reagent kit for preparing a cell sample, the kit containing a solid support, a trapping substance that traps a target cell and a cleavable linker capable of linking the solid support and the trapping substance, in which when the solid support and the trapping substance are linked by the linker, a fluorescent dye is bound to a site on the linker closer to the trapping substance than a cleavage position in the linker or a site on the trapping substance except for a binding site to the target cell in the trapping substance.

In the reagent for preparing a cell sample or the reagent kit for preparing a cell sample, the solid support may be a bead. Furthermore, in the reagent for preparing a cell sample or the reagent kit for preparing a cell sample, the bead may be a magnetic bead.

In the reagent for preparing a cell sample or the reagent kit for preparing a cell sample, the linker may be a DNA linker.

In the reagent for preparing a cell sample or the reagent kit for preparing a cell sample, the trapping substance may be a receptor-binding site, for example, an antibody or an antibody fragment.

In the reagent for preparing a cell sample or the reagent kit for preparing a cell sample, the fluorescent dye may be a fluorescent dye available for separating of a target cell by a cell sorter.

Furthermore, the present technology provides a method for preparing a cell sample for separating a target cell by a cell sorter using the reagent for preparing a cell sample or the reagent kit for preparing a cell sample according to the present technology. This method includes forming a composite of a reagent for preparing a cell sample of the present technology and a cell, separating the cell using a solid support contained in the reagent for preparing a cell sample, cleaving a linker contained in the reagent for preparing a cell sample and subjecting the resultant cell stained with a fluorescent dye as a cell sample for separating a target cell by a cell sorter.

### Effects of the Invention

According to the present technology, enrichment of a target cell using an antibody-modified solid support and subsequent staining for separating by a cell sorter can be carried out at the same time.

### BRIEF DESCRIPTION OF DRAWINGS

FIG. 1 is a flowchart schematically showing enrichment of a target cell by beads to separation of the target cell by a cell sorter.

FIG. 2 is a schematic view showing a configuration example (1) of a reagent for preparing a cell sample according to the present technology.

FIG. 3 is a schematic view showing a configuration example (2) of a reagent for preparing a cell sample according to the present technology.

### MODE FOR CARRYING OUT THE INVENTION

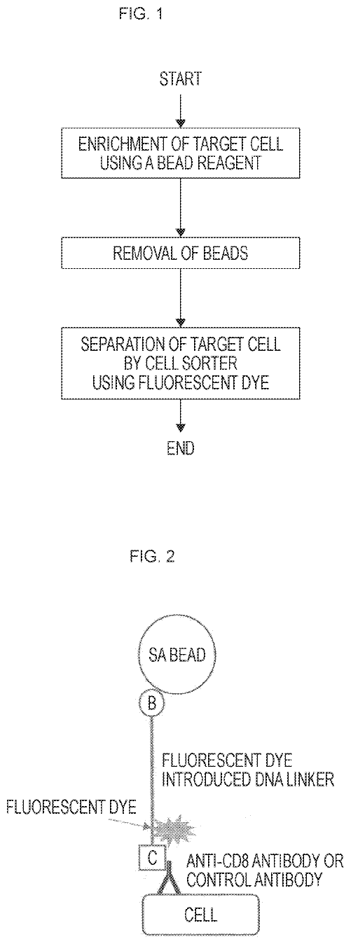

The reagent for preparing a cell sample according to the present technology includes a solid support and a trapping substance that is linked to the solid support by a cleavable linker and traps a target cell. The reagent for preparing a cell sample according to the present technology also includes a fluorescent dye. The fluorescent dye is bound to a site on the linker closer to the trapping substance than a cleavage position in the linker or a site on the trapping substance except for a binding site to the target cell in the trapping substance. Use of the reagent for preparing a cell sample enables enrichment of a target cell using the antibody-modified solid support and subsequent staining for separating by a cell sorter, at the same time. For example, in FIG. 1, as a pre-step for processing by a cell sorter, a target cell is enriched in a cell sample to be separated (hereinafter, referred to as a “separation target sample”). The target cell is enriched by an antibody-modified solid support, and then, the solid support is removed. In this manner, separation by the cell sorter can be smoothly performed using the fluorescent dye remaining in the proximity of the target cell. According to the present technology, since a target cell can be enriched and stained at the same time, it is possible to reduce the time required for preparing a cell sample. If the time required for preparing a cell sample is reduced, it is possible to prevent a decrease in cell-viability and suppress the cost required for producing a cell-based medicine.

The target cell may be any cell as long as it is wanted to separate. Examples of the cell may include animal cells (such as blood cells) and plant cells. The cell can be, in particular, a blood cell or a tissue cell. Examples of the blood cells include white blood cells (for example, peripheral blood mononuclear cells), red blood cells, and platelets. The blood cells include particularly white blood cells. Examples of the white blood cells include monocytes (macrophages), lymphocytes, neutrophils, basophils, and eosinophils. The cell may be, for example, a floating cell such as a T cells and a B cell. The tissue cell may be, for example, a cultured adherent cell or an adherent cell separated from a tissue. Furthermore, the cell may be a tumor cell. The cell may be a cultured cell or a cell not cultured. A desired bioparticle may be, for example, a cell for use in therapy or a blood cell such as a white blood cell. If the target cell is a blood cell, the separation target sample is a blood-derived sample, such as a thawed frozen apheresis sample, a fresh apheresis (unfrozen) sample, and a whole blood sample.

The trapping substance that traps a target cell may be, for example, a substance that itself binds to the target cell (also referred to as “target-cell binding substance”), or a substance that traps the target cell via another substance. In the latter case, the substance that traps a target cell itself does not bind to the cell, but the another substance may binds to the target cell.

The target-cell binding substance is a substance that itself binds to a target cell, as described above. The substance is, for example, an antibody or an antibody fragment, specifically an antibody or antibody fragment that binds to an antigen present on the surface of a target cell, and more specifically an antibody or antibody fragment that binds to a surface antigen of a cell.

The substance that traps a target cell via another substance may not be a substance that itself binds to a target cell, as described above. The substance that traps a target cell via another substance may be, for example, a substance that binds to a target-cell binding substance, such as a protein that binds to an antibody or an antibody fragment or a protein that specifically binds to an antibody or an antibody fragment. Examples of such a protein may include an antibody-binding protein, e.g., any one of Protein A, Protein G, Protein L, and Protein A/G, or any combination thereof. Examples of the protein further include an antibody or antibody fragment that binds to the antibody/antibody fragment to be used as a target-cell binding substance, streptavidin that binds to biotin previously bound to the antibody/antibody fragment to be used as a target-cell binding substance, and an anti-biotin antibody/antibody fragment.

In a preferred embodiment of the present technology, a receptor-binding site of, e.g., an antibody, is used as the target-cell binding substance. The receptor-binding site may be any site as long as it specifically binds to a receptor present on the surface of a target cell. Not only an antibody or an antibody fragment but also a peptide, a glycopeptide, a soluble receptor, a steroid, a hormone, a mitogen, an antigen, a super-antigen, a growth factor, a cytokine, a leptin, a viral protein, an adhesion molecule, a chemokine, or the like is acceptable.

In the reagent for preparing a cell sample of the present technology, in a case where a fluorescent dye binds to a trapping substance, the fluorescent dye binds to a site on the trapping substance except for the binding site to the target cell in the trapping substance. Such a binding site can be appropriately prepared by those skilled in the art in accordance with a specific trapping substance to be used.

The solid support is not particularly limited as long as the cell to which it binds and the cell to which it does not bind can be separated.

In a preferred embodiment of the present technology, a bead is used as the solid support. For example, in a case where separation is performed by a filter module capable of separating particles by size, beads having an appropriate size according to the threshold value of the filter module to be used may be used. Furthermore, if a magnetic bead is used, a target cell can be magnetically separated. Since various magnetic bead-products are commercially available and a functional group for binding to a linker vary depending on the product, selection may be appropriately made. For example, a commercially available product, Magnosphere (JSR) include products having, e.g., streptavidin, a carboxy group, and a tosyl group, as a functional group for binding to a linker. Furthermore, other products have an amino group and an azide group as the functional group for binding to a linker, for example. The binding to the linker can be performed by preparing the functional group of a linker so as to correspond to the functional group of a bead prepared.

As the solid support, a support having a shape (i.e., neither spherical nor elliptical) other than a bead shape can be used. Examples of such a support include a fibrous support and a pillar support to be provided in a column or a channel. These supports can bind to a linker and can be immobilized in advance on the inner wall in a predetermined region of a column or a channel. Then, when a sample containing a target cell passes through the column or the channel, the composite of the reagent for preparing a cell sample (i.e., [solid support]-[linker]-[trapping substance]) of the present technology and the target cell is formed in a state of being immobilized on the inner wall of the column or the channel, whereas the other cells are removed without being immobilized. In this manner, the target cell can be separated from the other cells. Thereafter, the target cells immobilized on the inner wall of the column or channel can be collected by cleaving the linker.

The linker is not particularly limited as long as it is “cleavable”, that is, can be cut by some means. The linker can be selected in combination with its cleavage means. However, it is preferable that the linker is cleavable by a treatment that does not adversely affect the other useful substances present in the system, such as a target cell, a fluorescent dye, and a trapping substance. For example, a linker cleavable by enzyme treatment is preferred.

As the linker cleavable by enzyme treatment, for example, a nucleic-acid linker can be suitably used. The nucleic-acid linker can be cleaved with an endonuclease. Enzymes such as DNase (for example, DNase I, DNase II, Nuclease P1, Turbonuclease, Benzonase), RNase, and a restriction enzyme can be selectively used depending on the type of nucleic acid. The nucleic acid may be DNA or RNA but DNA is more preferable in view of synthesis cost and resistance to degradation. Furthermore, the chain length of the nucleic-acid linker is not particularly limited. However, in consideration of, e.g., the synthesis cost and the distance between a solid support and a trapping substance, the chain length is preferably 10 to 200 residues, more preferably 15 to 100 residues, and still more preferably 15 to 50 residues.

In the reagent for preparing a cell sample of the present technology, in a case where a fluorescent dye binds to a linker, the fluorescent dye binds to a site on the linker closer to the trapping substance than the cleavage position in the linker. Here, even if the cleavage position cannot be clearly specified, such as a case where the single-stranded DNA linker is cleaved with DNase, if a fluorescent dye binds to a site on the linker closer to the trapping substance than the cleavage position up to a certain ratio, it should be understood that the case corresponds to the aforementioned case: “a fluorescent dye binds to a site on the linker closer to the trapping substance than the cleavage position in the linker”. Furthermore, if the binding site of a fluorescent dye is arranged to be closer to the trapping substance on the linker, the percentage of a case where the binding site of the fluorescent dye is closer to the trapping substance than the cleavage position of the linker can be increased. Moreover, the phosphodiester bond around a fluorescent dye may be cleaved and replaced with a phosphorothioate group such that a fluorescent dye rarely cleaved with a nucleolytic enzyme.

Moreover, it is also possible to accurately specify the cleavage position on a nucleic-acid linker. For example, a double stranded nucleic-acid linker is employed and the recognition sequence by a restriction enzyme is inserted in the sequence. If so, the nucleic-acid linker (for example, DNA linker) can be accurately cleaved at the site of the recognition sequence by the restriction enzyme. Alternatively, using RNase H, which selectively cleaves RNA in an RNA-DNA double strand, the RNA portion in the RNA-DNA double-stranded nucleic-acid linker can be accurately cleaved.

Note that in a case where such a double-stranded nucleic-acid linker is used, if one of the strands of the double-stranded nucleic acid is bound to a solid support and the other strand is bound to a trapping substance, a composite of [solid support]-[double-stranded nucleic-acid linker]-[trapping substance] can be easily formed by hybridization of these nucleic acids.

The term “fluorescent dye” herein refers to a substance or a portion thereof (e.g., a functional group) capable of emitting fluorescence in normal assay conditions using cells. Examples of such a fluorescent dye include a fluorescein dye (e.g., FITC), a cyanine dye such as Cy3 or Cy5, and TAMRA. Since these are commercially available as monomers for synthesizing DNA, they are suitable for being bound onto a nucleic-acid linker. As the fluorescent dye, further VioBlue or Alexa Fluor-series dyes can be used. Furthermore, other than so-called dyes, a modifying group (for example, a cholesterol group, a stearyl group, a photo-cleavable PC linker) available as a monomer for synthesizing DNA can be introduced. As described above, these modifying groups are also included in the “fluorescent dye” of the present technology. Furthermore, the number of fluorescent dyes to be introduced can also be increased, and a plurality of fluorescent dyes can be introduced using a technique, for example, KIRAVIA Dyes.

A preferred embodiment of the present technology is shown in FIG. 2. In FIG. 2, B represents biotin and C represents a carboxy group, both of which are introduced in a DNA linker. In this example, a streptavidin-modified bead is bound via biotin and an antibody is bound via a carboxy group. In FIG. 2, a fluorescent dye is bound to a site on the DNA linker close to the antibody.

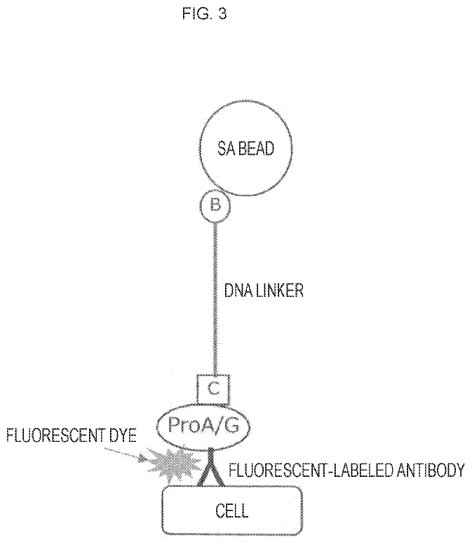

Furthermore, another preferred embodiment is shown in FIG. 3. The difference from FIG. 2 is that protein A/G is present between the antibody and the DNA linker, and a fluorescent dye is bound to the antibody.

The reagent for preparing a cell sample of the present technology may be formed in a place not touched by a human hand, such as a closed channel. Thus, the reagent may be provided as a kit containing individual members separately. Therefore, according to another aspect, the present technology provides a reagent kit for preparing a cell sample, the kit containing a solid support, a trapping substance that traps a target cell and a cleavable linker capable of linking the solid support and the trapping substance, in which when the solid support and the trapping substance are linked by the linker, a fluorescent dye is bound to a site on the linker closer to the trapping substance than a cleavage position in the linker or a site on the trapping substance except for a binding site to the target cell in the trapping substance.

Furthermore, the present technology provides a method for preparing a cell sample for separating a target cell by a cell sorter using the reagent for preparing a cell sample or the reagent kit for preparing a cell sample according to the present technology. This method includes forming a composite of a reagent for preparing a cell sample of the present technology and a cell, separating the cell using a solid support contained in the reagent for preparing a cell sample, cleaving a linker contained in the reagent for preparing a cell sample and subjecting the resultant cell stained with a fluorescent dye as a cell sample for separating a target cell by a cell sorter.

The reagent for preparing a cell sample, reagent kit for preparing a cell sample, and method for preparing a cell sample according to the present technology can also employ the following constitutions.

(1)

A reagent for preparing a cell sample, the reagent containing a solid support and a trapping substance that is linked to the solid support by a cleavable linker and traps a target cell, in which a fluorescent dye is bound to a site on the linker closer to the trapping substance than a cleavage position in the linker or a site on the trapping substance except for a binding site to the target cell in the trapping substance.

(2)

The reagent for preparing a cell sample according to (1), in which the solid support is a bead.

(3)

The reagent for preparing a cell sample according to (2), in which the bead is a magnetic bead.

(4)

The reagent for preparing a cell sample according to any one of (1) to (3), in which the linker is a DNA linker.

(5)

The reagent for preparing a cell sample according to any one of (1) to (4), in which the trapping substance is a receptor-binding site.

(6)

The reagent for preparing a cell sample according to any one of (1) to (4), in which the trapping substance is an antibody or an antibody fragment.

(7)

The reagent for preparing a cell sample according to any one of (1) to (6), in which the fluorescent dye is a fluorescent dye available for separating a target cell by a cell sorter.

(8)

A reagent kit for preparing a cell sample, the kit containing a solid support, a trapping substance that traps a target cell, and a cleavable linker capable of linking the solid support and the trapping substance, in which when the solid support and the trapping substance are linked by the linker, a fluorescent dye is bound to a site on the linker closer to the trapping substance than a cleavage position in the linker or a site on the trapping substance except for a binding site to the target cell in the trapping substance.

(9)

The reagent kit for preparing a cell sample according to (8), in which the solid support is a bead.

(10)

The reagent kit for preparing a cell sample according to (9), in which the bead is a magnetic bead.

(11)

The reagent kit for preparing a cell sample according to any one of (8) to (10), in which the linker is a DNA linker.

(12)

The reagent kit for preparing a cell sample according to any one of (8) to (11), in which the trapping substance is a receptor-binding site.

(13)

The reagent kit for preparing a cell sample according to any one of (8) to (11), in which the trapping substance is an antibody or an antibody fragment.

(14)

The reagent kit for preparing a cell sample according to any one of (8) to (13), in which the fluorescent dye is a fluorescent dye available for separating a target cell by a cell sorter.

(15)

A method for preparing a cell sample for separating a target cell by a cell sorter, using the reagent for preparing a cell sample according to any one of (1) to (7) or the reagent kit for preparing a cell sample according to any one of (8) to (14), the method including:
(a) a step of forming a composite of the reagent for preparing a cell sample and a cell;
(b) a step of separating the cell using a solid support contained in the reagent for preparing a cell sample;
(c) cleaving a linker contained in the reagent for preparing a cell sample; and
(d) a step of using the cell stained with a fluorescent dye and obtained in the step (c) as a cell sample for separating a target cell by a cell sorter.

### EXAMPLES

Embodiments of the present technology will be specifically described on the basis of the following examples, but the present technology is not limited to these examples.

### Example 1: Trapping of Target Cell by Antibody-Immobilized Beads Via FITC-Introduced DNA Linker (1)

### 1. Outline of the Example

In this example, an anti-CD8 antibody was used as the antibody to be immobilized on beads and an experiment was performed as follows.
(i) Staining of cell sample with antibody (CD3-PE, CD4-APC) commonly used for flow cytometer (cell sample: PBMCs);
(ii) Antibody-immobilized magnetic beads via FITC-introduced DNA linker are reacted with the cells stained in the above (antibody: anti-CD8 antibody, or isotype control antibody (negative control));
(iii) Magnetically separating the beads, followed by measuring the cells remaining in the supernatant by a flow cytometer (If target CD8-positive cells are trapped by beads, it is expected that the ratio of CD4-positive cells in the supernatant increases) (CD3-positive cells are roughly divided into either CD4-positive cells or CD8-positive cells. The ratio is already determined depending on the sample);
(iv) Separating the cells trapped by the beads and measuring them similarly by a flow cytometer (FITC-introduced DNA linker portion between a bead and the antibody is cleaved with DNase I to separate the cell).

### 2. Preparation of Antibody-Immobilized Beads for Trapping Cells

### (i) Binding of Antibody to DNA Linker

Synthetic oligo-DNA having the following sequence was prepared by introducing a carboxy group into the 5′ end, biotin into the 3′ end, and a fluorescent dye in the proximity of the 5′ end.

Sequence 1:
(nucleotide sequence: SEQ ID NO: 1)
cfCCATCTTTCCGCATCAACGAATATGTTAGCb
Sequence 2:
(nucleotide sequence: SEQ ID NO: 2)
cTCfCCATCTTTCCGCATCAACGAATATGTTAGCb
[c: 5′-Carboxy C10, f: FITC-dT, b: 3′-BiotinTEG]

The carboxy group of the synthetic oligo-DNA was activated with EDC and NHS (NHS esterification), and then bound to the antibody. Specifically, synthetic oligo-DNA, 1-ethyl-3-(3-dimethylaminopropyl) carbodiimide hydrochloride (EDC) and N-hydroxysuccinimide (NHS) were added to MES buffer (pH5.4), and the mixture was allowed to react at room temperature for 30 minutes to 1 hour. Thereafter, purification was performed using a Microspin G-25 column (Cytiva). To the eluate from the column (purified NHS-esterified synthetic oligo-DNA), HEPES buffer (pH7.9) and various types of antibodies were added, and the mixture was allowed to react at room temperature for 1 to 2 hours. Thereafter, washing was performed using Ultracel-30K (Merck Millipore) and a synthetic oligo-DNA-modified antibody was collected.

### (ii) Immobilization on Streptavidin-Modified Beads

A synthetic oligo-modified antibody was immobilized on Magnosphere MS300/Streptavidin (JSR). Specifically, Magnosphere was placed in an Eppendorf tube and magnetically separated. After the solvent was exchanged with PBS buffer, the synthetic oligo-modified antibody prepared in the above (i) was added. The mixture was allowed to react at room temperature for 30 minutes to 1 hour. The operation of exchanging the solvent with PBS buffer by magnetic separation was repeated three times.

### 3. Confirmation of Target-Cell Trapping

(i) Staining PBMCs (CTL) with PE-Labeled Anti-CD3 Antibody and APC-Labeled Anti-CD4 Antibody (Both were Provided by Biolegend) (the Number of Cells: About 4.2×10⁵)

These were incubated at 4° C. for 15 to 20 minutes and washed with PBS buffer containing 0.5% BSA.

(ii) Reaction of the Antibody-Immobilized Magnetic Beads Via the FITC-Introduced DNA Linker with the Cells Mentioned Above, Followed by Magnetic Separation of the Bead to Separate the Supernatant and the Beads

The antibody-immobilized magnetic beads (about 1.5×10⁶) and the cells were reacted at room temperature for 30 minutes to 1 hour with gentle stirring. As the antibody, an anti-CD8 antibody or an Isotype Control antibody (both were provided by Biolegend) was used.

(iii) The Cells Remaining in the Supernatant were Measured by a Flow Cytometer.

A gate was applied to the singlet of lymphocytes to calculate the ratio of CD3-positive and CD4-positive cells and CD3-positive and CD4-negative cells in each sample (Table 1 below). As is apparent from Table 1, the ratio of CD4-positive cells increased in the sample of the anti-CD8 antibody-immobilized beads.

(iv) Measurement of the Cells Trapped by the Beads and Measurement of them by a Flow Cytometer.

After the concentration of DNase I (Invitrogen) was adjusted to 2000 U/mL, and incubation was performed at room temperature for 30 minutes to 1 hour with gentle stirring, the beads were magnetically separated, and the cells separated were measured. The numbers of detected cells in the lymphocyte fractions during a certain period of time were compared between individual beads. It was confirmed that cells were trapped by the beads (Table 2 below). Cells were detected also from the beads on which the control antibody was immobilized. This was considered to be due to nonspecific adsorption. Furthermore, a gate was applied to the singlet of lymphocytes to compare the fluorescence intensity of FITC and an unstained sample. It was also confirmed that they were trapped and simultaneously stained with FITC (Table 2 below).

TABLE 1
Table 1: Ratio of cells remaining in the supernatant after
cells are trapped by antibody-immobilized beads

| | Ratio of CD3- | Ratio of CD3- |
|---|---|---|
| | positive and CD4- | positive and CD4- |
| Sample | positive cells | negative cells |
| Before addition of beads | 72% | 28% |
| After addition of | 67% | 33% |
| control antibody | | |
| immobilized beads | | |
| After addition of anti- | 93% | 7% |
| CD8 antibody immobilized | | |
| beads | | |

TABLE 2
Table 2: Measurement results after separation of cells
trapped by beads

| | Number of cells | |
|---|---|---|
| Antibody immobilized | detected in | Ratio of FITC- |
| on beads | lymphocyte fraction | positive cells |
| Control antibody | 1687 | 19% |
| Anti-CD8 antibody | 7256 | 74% |

### Example 2: Trapping of Target Cell by Antibody-Immobilized Beads Via FITC-Introduced DNA Linker (2)

### 1. Outline of the Example

The same procedure as in Example 1 was repeated except that the antibody to be immobilized on the beads was changed from the anti-CD8 antibody to an anti-CD4 antibody. The outline of the Example is as follows.
(i) Staining a cell sample with an antibody (CD3-PE, CD8-APC) commonly used for a flow cytometer (cell sample: PBMC);
(ii) Reacting antibody-immobilized magnetic beads via an FITC-introduced DNA linker with the cells stained above (antibody: anti-CD4 antibody, or isotype control antibody (negative control));
(iii) Magnetically separating the beads followed by measuring the cells remaining in the supernatant by a flow cytometer (If target CD4-positive cells are trapped by the beads, it is expected that the ratio of CD8-positive cells in the supernatant increases) (CD3-positive cells are roughly divided into either CD4-positive or CD8-positive cells. The ratio is determined depending on the sample);
(iv) Separating the cells trapped by the beads and measuring them similarly by a flow cytometer (FITC-introduced DNA linker portion between a bead and the antibody is cleaved with DNase I to separate the cell).

### 2. Preparation of Antibody-Immobilized Beads for Trapping Cells

Antibody-immobilized beads for trapping cells were prepared in the similar manner as in Example 1.

### 3. Confirmation of Target-Cell Trapping

(i) Staining of PBMCs (CTL) with PE-Labeled Anti-CD3 Antibody and APC-Labeled Anti-CD8 Antibody (Both were Provided by Biolegend) (the Number of Cells: About 2.8×10⁵)

These were incubated at 4° C. for 15 to 20 minutes and washed with PBS buffer containing 0.5% BSA.

(ii) Reaction of the Antibody-Immobilized Magnetic Beads Via the FITC-Introduced DNA Linker with the Cells Mentioned Above, Followed by Magnetic Separation of the Bead to Separate the Supernatant and the Beads

The antibody-immobilized magnetic beads (about 1.5×10⁶) and the cells were reacted at room temperature for 30 minutes to 1 hour with gentle stirring. As the antibody, an anti-CD4 antibody or an Isotype Control antibody (both were provided by Biolegend) was used.

(iii) The Cells Remaining in the Supernatant were Measured by a Flow Cytometer.

A gate was applied to the singlet of lymphocytes to calculate the ratio of CD3-positive and CD8-positive cells and CD3-positive and CD8-negative cells in each sample (Table 3 below). As is apparent from Table 3, the ratio of CD8-positive cells increased in the sample of the anti-CD4 antibody-immobilized beads.

(iv) Measurement of the Cells Trapped by the Beads and Measurement of them by a Flow Cytometer.

After the concentration of DNase I (Invitrogen) was set at 500 U/mL, and incubation was performed at room temperature for about 1.5 hours with gentle stirring, the beads were magnetically separated, and the cells separated were measured. The numbers of detected cells in the lymphocyte fraction during a certain period of time were compared between individual beads. It was confirmed that cells were trapped by the beads (Table 4 below). Cells were detected also from the beads on which the control antibody was immobilized. This was considered to be due to nonspecific adsorption. Furthermore, a gate was applied to the singlet of lymphocytes to compare the fluorescence intensity of FITC and an unstained sample. It was confirmed that they were trapped and simultaneously stained with FITC (Table 4 below). In the Example, the reason why the ratio of FITC-positive cells was low was considered as the effect of longer incubation time with DNase I.

TABLE 3
Table 3: The ratio of cells remaining in the supernatant
after cells were trapped by antibody-immobilized beads

| | Ratio of CD3- | Ratio of CD3- |
|---|---|---|
| | positive and CD8- | positive and CD8- |
| Sample | positive cells | negative cells |
| Before addition of beads | 18% | 82% |
| After addition of control antibody | 19% | 81% |
| immobilized beads | | |
| After addition of anti-CD4 antibody | 69% | 31% |
| immobilized beads | | |

TABLE 4
Table 4: Measurement results after separation of cells
trapped by beads

| | Number of cells | Ratio of FITC- |
|---|---|---|
| Antibody immobilized | detected in | positive cells |
| on beads | lymphocyte fraction | |
| Control antibody | 1407 | 26% |
| Anti-CD4 antibody | 8222 | 50% |

### Example 3: Trapping of Target Cell by Beads Immobilized on VioBlue-Labeled Antibody Via DNA Linker

### 1. Outline of the Example

In the example, unlike Examples 1 and 2, a fluorescent dye was not introduced into the DNA linker, and an antibody labeled with a fluorescent dye (VioBlue) that is used for cell staining in e.g., a flow cytometer, was used.

### 2. Preparation of Antibody-Immobilized Beads for Trapping Cells

### (i) Binding of Protein A/G and DNA Linker

A synthetic oligo-DNA having the following sequence was prepared by introducing a carboxy group into the 5′ end and biotin into the 3′ end.

Sequence 3:
(nucleotide sequence: SEQ ID NO: 3)
cCCATCTTTCCGCATCAACGAATATGTTAGCb
[c: 5′-Carboxy C10, b: 3′ BiotinTEG]

After the carboxy group of the synthetic oligo-DNA was activated with EDC and NHS (NHS esterification), the synthetic oligo-DNA was bound to Protein A/G. Specifically, synthetic oligo-DNA, 1-ethyl-3-(3-dimethylaminopropyl) carbodiimide hydrochloride (EDC) and N-hydroxysuccinimide (NHS) were added to MES buffer (pH5.4), and the mixture was allowed to react at room temperature for 30 minutes to 1 hour. Thereafter, purification was performed using a Microspin G-25 column (Cytiva), and HEPES buffer (pH7.9) and Protein A/G (BioVision) were added to the eluate from the column (purified NHS-esterified synthetic oligo-DNA). The mixture was allowed to react at room temperature for 1 to 2 hours. Thereafter, washing was performed using Ultracel-30K (Merck Millipore) and a synthetic oligo-DNA-modified Protein A/G was collected.

### (ii) Immobilization of Synthetic Oligo-DNA Modified Protein A/G on Streptavidin-Modified Beads and Binding of Antibody and Protein A/G Moiety

A synthetic oligo-modified Protein A/G was immobilized on Magnosphere MS300/Streptavidin (JSR). Specifically, Magnosphere was placed in an Eppendorf tube, magnetically separated, and the solvent was exchanged with PBS buffer. Thereafter, the synthetic oligo-modified Protein A/G prepared in the above (i) was added. The mixture was allowed to react at room temperature for 30 minutes to 1 hour. The operation of exchanging the solvent with PBS buffer by magnetic separation was repeated three times. Various types of antibodies were added thereto, and the mixture was allowed to react at room temperature for 30 minutes to 1 hour. Similarly, washing by magnetic separation was repeated three times.

### 3. Confirmation of Target-Cell Trapping

(i) Staining of Immuno-Trol (Beckman Coulter) with PE-Labeled Anti-CD3 Antibody and APC-Labeled Anti-CD4 Antibody (Both were Provided by Biolegend) (the Number of White Blood Cells: About 2.5×10⁵).

These were incubated at 4° C. for 15 to 20 minutes and washed with PBS buffer containing 0.5% BSA.

### (ii) Reaction of the Antibody-Immobilized Magnetic Beads and the Cells, Followed by Magnetic Separation of the Bead to Separate the Supernatant and the Beads

The antibody-immobilized magnetic beads (about 1.5×10⁶) and the cells were reacted at room temperature for 30 minutes to 1 hour with gentle stirring. As the antibody, a VioBlue-labeled anti-CD8 antibody (Miltenyi) or a Control antibody (Biolegend) was used.

(iii) The Cells Remaining in the Supernatant were Measured by a Flow Cytometer.

A gate was applied to the singlet of lymphocytes to calculate the ratio of CD3-positive and CD4-positive cells and CD3-positive and CD4-negative cells in each sample (Table 5 below). As is apparent from Table 5, the ratio of CD4-positive cells increased in the sample of the anti-CD8 antibody-immobilized beads.

(iv) Measurement of the Cells Trapped by the Beads and Measurement of them by a Flow Cytometer.

After the concentration of DNase I (Invitrogen) was set at 1000 U/mL, and incubation was performed at room temperature for about 1.5 hours with gentle stirring, the beads were magnetically separated, and measured. The number of detected cells in the lymphocyte fraction during a certain period of time was compared between individual beads. It was confirmed that cells were trapped by the beads (Table 6 below). Cells were detected also from the beads on which the control antibody was immobilized. This was considered to be due to nonspecific adsorption. Furthermore, with respect to the cells detected in the lymphocyte fraction, fluorescence intensity was compared between the unstained sample and VioBlue. It was confirmed that the cells were trapped and simultaneously stained with VioBlue (Table 6 below). In the Example, the control antibody is not fluorescently labeled.

TABLE 5
Table 5: The ratio of cells remaining in the supernatant
after cells were trapped by antibody-immobilized beads

| | Ratio of CD3- | Ratio of CD3- |
|---|---|---|
| | positive and CD4- | positive and CD4- |
| Sample | positive cells | negative cells |
| Before addition of beads | 59% | 41% |
| After addition of control antibody | 61% | 39% |
| immobilized beads | | |
| After addition of | 74% | 26% |
| VioBlue label/anti-CD8 | | |
| antibody immobilized | | |
| beads | | |

TABLE 6
Table 6: Measurement results after separation of cells
trapped by beads

| | Number of cells | |
|---|---|---|
| Antibody immobilized | detected in | Ratio of VioBlue- |
| on beads | lymphocyte fraction | positive cells |
| Control antibody | 568 | — |
| VioBlue-labeled | 1955 | 84% |
| anti-CD8 antibody | | |

1. A reagent for preparing a cell sample, the reagent comprising a solid support and a trapping substance that is linked to the solid support by a cleavable linker and traps a target cell, wherein a fluorescent dye is bound to a site on the linker closer to the trapping substance than a cleavage position in the linker or a site on the trapping substance except for a binding site to the target cell in the trapping substance.

2. The reagent for preparing a cell sample according to claim 1, wherein the solid support is a bead.

3. The reagent for preparing a cell sample according to claim 2, wherein the bead is a magnetic bead.

4. The reagent for preparing a cell sample according to claim 1, wherein the linker is a DNA linker.

5. The reagent for preparing a cell sample according to claim 1, wherein the trapping substance is a receptor-binding site.

6. The reagent for preparing a cell sample according to claim 1, wherein the trapping substance is an antibody or an antibody fragment.

7. The reagent for preparing a cell sample according to claim 1, wherein the fluorescent dye is a fluorescent dye available for separating a target cell by a cell sorter.

8. A reagent kit for preparing a cell sample, the kit comprising a solid support, a trapping substance that traps a target cell, and a cleavable linker capable of linking the solid support and the trapping substance, wherein when the solid support and the trapping substance are linked by the linker, a fluorescent dye is bound to a site on the linker closer to the trapping substance than a cleavage position in the linker or a site on the trapping substance except for a binding site to the target cell in the trapping substance.

9. The reagent kit for preparing a cell sample according to claim 8, wherein the solid support is a bead.

10. The reagent kit for preparing a cell sample according to claim 9, wherein the bead is a magnetic bead.

11. The reagent kit for preparing a cell sample according to claim 8, wherein the linker is a DNA linker.

12. The reagent kit for preparing a cell sample according to claim 8, wherein the trapping substance is a receptor-binding site.

13. The reagent kit for preparing a cell sample according to claim 8, wherein the trapping substance is an antibody or an antibody fragment.

14. The reagent kit for preparing a cell sample according to claim 8, wherein the fluorescent dye is a fluorescent dye available for separating a target cell by a cell sorter.

15. A method for preparing a cell sample for separating a target cell by a cell sorter using the reagent for preparing a cell sample according to claim 1, the method comprising:
(a) a step of forming a composite of the reagent for preparing a cell sample and a cell;
(b) a step of separating the cell using a solid support contained in the reagent for preparing a cell sample;
(c) cleaving a linker contained in the reagent for preparing a cell sample; and
(d) a step of using the cell stained with a fluorescent dye and obtained in the step (c) as a cell sample for separating a target cell by a cell sorter.
